(19) United States
(12) Patent Application Publication
(10) Pub. No.: US 2023/0201128 A1
(43) Pub. Date: Jun. 29, 2023

(54) METHOD OF TREATING OBESITY-INDUCED GLUCOSE INTOLERANCE AND LIVER FIBROSIS

(71) Applicants: THE TRUSTEES OF COLUMBIA UNIVERSITY IN THE CITY OF NEW YORK, New York, NY (US); THE REGENTS OF THE UNIVERSITY OF CALIFORNIA, Oakland, CA (US)

(72) Inventors: Utpal PAJVANI, New York, NY (US); Li QIANG, Closter, NJ (US); Zhen GU, Oakland, CA (US)

(21) Appl. No.: 17/999,384

(22) PCT Filed: May 19, 2021
(86) PCT No.: PCT/US2021/033184

§ 371 Date: Nov. 18, 2022

Related U.S. Application Data
(60) Provisional application No. 63/027,451, filed on May 20, 2020.

(51) Int. Cl. A61K 9/51; A61K 31/55; A61P 3/10; A61P 1/16

(52) U.S. Cl. CPC A61K 9/5153; A61K 31/55; A61P 3/10; A61P 1/16

(57) ABSTRACT

The method of treating obesity-induced glucose intolerance and liver fibrosis includes administering to a patient a therapeutically effective amount of a substance for inhibiting the Notch signaling pathway of the patient. The substance is formed from encapsulated γ-secretase inhibitor (GSI) nanoparticles. As a non-limiting example, each nanoparticle may be formed from a GSI, such as dibenzazepine (a bioavailable GSI) encapsulated in poly(lactic co-glycolic acid) (PLGA). The encapsulated GSI nanoparticles (GSI NPs) provide localized and effective inhibition of hepatic Notch signaling, thus improving obesity-induced glucose tolerance and liver fibrosis without intestinal side effects.

### CROSS-REFERENCE TO RELATED APPLICATION

This application claims the benefit of U.S. Provisional Patent Application No. 63/027,451, filed on May 20, 2020.

### GOVERNMENT LICENSE RIGHTS

This invention was made with government support under Grant No. RO1 DK103818, awarded by the National Institutes of Health; Grant No. RO1 DK119767, awarded by the National Institutes of Health; and Grant No. T32 DK065522, awarded by the National Institutes of Health. The government has certain rights in the invention.

### TECHNICAL FIELD

The disclosure of the present patent application relates to the treatment of obesity-induced glucose intolerance and liver fibrosis, and particularly to a treatment involving administration of encapsulated γ-secretase inhibitor (GSI) nanoparticles to a patient.

### BACKGROUND ART

With the rise of obesity worldwide, the prevalence of co-morbidities such as type 2 diabetes mellitus (T2DM), non-alcoholic fatty liver disease (NAFLD), and non-alcoholic steatohepatitis (NASH) are also increasing at alarming rates. While the mechanisms by which obesity leads to its metabolic complications are still being investigated, the resulting demand for therapeutic interventions continues to be largely unmet. Despite numerous new pharmacologic entries to the market, T2DM remains the most common cause of end-stage renal disease and blindness in the United States, and a major contributor to mortality. Patients with NASH have even fewer options; without FDA-approved pharmacotherapy, the only effective treatment is liver transplantation.

In obesity, excess lipid accumulation contributes to insulin resistance, which manifests in the liver as increased forkhead box protein O1 (FOXO1)-dependent hepatic glucose production (HGP). In the absence of insulin, unphosphorylated FOXO1 binds to the insulin response element to promote transcription of glucose-6-phosphatase (G6PC) and phosphoenolpyruvate carboxykinase (PCK1), encoding the rate-limiting enzymes in glycogenolysis and gluconeogenesis, respectively. Upon insulin presence and activation of PI3K/AKT, FOXO1 is phosphorylated and excluded from the nucleus, reducing HGP. This signaling is disrupted in the insulin-resistant state, where FOXO1 is constitutively active.

In investigating the effects of FOXO1 on T2DM pathophysiology, a “re-activation” of the Notch signaling pathway in hepatocytes was uncovered. Notch is an evolutionarily conserved cell signaling system that directs embryonic development. Ligand binding induces γ-secretase-mediated Notch receptor cleavage, releasing the Notch intracellular domain (NICD). NICD translocates into the nucleus where it binds to mastermind-like protein 1 (MAML1) and the principle Notch effector, recombining binding protein suppressor of hairless (RBP-JK). This complex promotes transcription of canonical Notch target genes, including the basic Helix-Loop-Helix (bHLH) hairy and enhancer of split (HES) and hairy/enhancer-of-split related with YRPW motif-like protein (HEYL) gene families that direct cell-fate decisions. In liver development, increased hepatoblast Notch activity directs differentiation into cholangiocytes and formation of bile ducts, with repression in adjacent cells leading to a hepatocyte lineage specification. Quiescent hepatocyte Notch activity persists into adulthood under typical physiologic conditions. However, in obese mice and patients, aberrant hepatocyte Notch activity that associated with the presence of T2DM, as well as biochemical (plasma transaminase) and histological (NAFLD activity score) readouts of NASH, were observed. Consistently, hepatocyte-specific Notch loss-of-function mice show attenuated diet-induced liver pathology, including improved glucose intolerance in mice fed a obesogenic high-fat diet (HFD), as well as reduced liver fibrosis in mice fed a novel NASH-provoking diet, whereas forced hepatocyte Notch activity accelerated diet effects in both models.

These data suggest that pharmacologic Notch inhibitors would have metabolic benefits. The best-studied Notch antagonists are small molecule inhibitors of the γ-secretase complex (GSI) that prevent activating cleavage of Notch receptors, and are presently in clinical trials for cancer. Indeed, GSI treatment reduced liver Notch activity, improved glucose metabolism, and ameliorated NASH diet-induced liver fibrosis, but simultaneously caused goblet cell metaplasia related to intestinal Notch inhibition.

Based on these data, it would clearly be desirable to develop liver-specific GSI administration, thus avoiding intestinal side effects while retaining therapeutic potency. Nanomedicine-mediated treatment allows for enhanced targeted drug delivery, and many nanoparticle compositions, e.g., poly(lactic co-glycolic acid) (PLGA), have favorable biodegradability and biocompatibility profiles. In order to be able to develop liver-specific GSI administration, it would be deisrable to be able to make use of nanoparticles as a therapeutic delivery agent. Thus, a method of treating obesity-induced glucose intolerance and liver fibrosis solving the aforementioned problems is desired.

### DISCLOSURE

The method of treating obesity-induced glucose intolerance and liver fibrosis includes administering to a patient a therapeutically effective amount of a substance for inhibiting the Notch signaling pathway of the patient. The substance is formed from encapsulated γ-secretase inhibitor (GSI) nanoparticles. As a non-limiting example, each nanoparticle may be formed from a GSI, such as dibenzazepine (a bioavailable GSI) encapsulated in poly(lactic co-glycolic acid) (PLGA). The encapsulated GSI nanoparticles (GSI NPs) provide localized and effective inhibition of hepatic Notch signaling, thus improving obesity-induced glucose tolerance and liver fibrosis without intestinal side effects, while retaining therapeutic potency in the liver. Additionally, PLGA provides favorable biodegradability and biocompatibility profiles. The method of treating obesity-induced glucose intolerance and liver fibrosis has been validated in a murine model of diet-induced obesity and glucose tolerance, which demonstrated a 34-58% reduction in Notch target gene expression.

These and other features of the present subject matter will become readily apparent upon further review of the following specification.

### BRIEF DESCRIPTION OF THE DRAWINGS

FIG. 1 schematically illustrates action of poly(lactic co-glycolic acid) (PLGA) encapsulated γ-secretase inhibitor (GSI) nanoparticles (GSI NPs) in the liver.

FIG. 2A is a graph showing size distribution of the GSI NPs.

FIG. 2B is a scanning electron microscope (SEM) image of the GSI NPs, showing their shapes (scale bar: 0.1 μm).

FIG. 2C is a plot of GSI NP size as a function of time, particularly showing that hydrodynamic nanoparticle size remained stable over one week.

FIG. 2D is a plot showing dissociation of the nanoparticles measured by GSI concentration over time.

FIG. 2E shows fluorescent signal images demonstrating that the PLGA nanoparticles are distributed mainly in the liver (L) and kidneys (K) at 1 h and 24 h post-tail vein injection (in C57BJ/6 male mice), with other tissues (e.g., heart (H), lungs (Lu), and spleen (S)) not showing significant nanoparticle accumulation at 24 h. n=3.

FIG. 3A shows the abundant liver Cy5.5 signal in a control group of mice.

FIG. 3B shows the abundant liver Cy5.5 signal in a group of mice treated with GSI NPs, shown four days post-injection (1 s exposure).

FIG. 3C liver sections from an untreated animal (1 s exposure).

FIG. 3D shows a small intestine section from GSI NP-treated mice (5 s exposure). Bright field images provided for visualization of tissue architecture (scale bars=200 μm).

FIG. 3E is a graph illustrating liver notch target gene expression after a single GSI NP IV injection; *p<0.05 for Control vs. GSI 2 Day. #p<0.05 for Control vs. GSI 4 Day, n=5/group.

FIG. 4A diagrammatically illustrates an injection schedule for testing of the GSI NPs.

FIG. 4B is a graph showing GSI NP-induced reduction in glucose excursion.

FIG. 4C is a graph showing glucose area under the curve (AUC) after intraperitoneal glucose administration.

FIG. 4D is a graph showing, at sacrifice of the experimental mice, GSI NP-induced reduced fasting blood glucose.

FIG. 4E is a graph showing body weight measurement corresponding to FIG. 4D.

FIG. 4F is a graph showing body composition measurement corresponding to FIG. 4D.

FIG. 4G is a graph showing liver and adipose tissue weight measurements corresponding to FIG. 4D.

FIG. 4H is a graph illustration GSI NP-induced reduction of Notch targets and expression of FOXO1 and its target genes.

FIG. 4I is a plot showing reduced liver FOXO1 and p-FOXO1^{S256} levels corresponding to FIG. 4H; *p<0.05, **p<0.01 as compared to control NP-treated mice, n=11-12/group.

FIG. 5A shows normal intestinal goblet cell number in GSI NP-treated mice by quantification of PAS+ cells (scale bar: 200 μm).

FIG. 5B shows representative confocal images of Cy5.5 signal in liver and extra-hepatic tissues taken from the same animal, and quantification of signal relative to liver (scale bar: 200 μm); *** p<0.001 as compared to liver Cy5.5 signal, n=10 (a) and 11-12/group.

FIG. 5C is a graph illustrating unchanged Notch target gene expression in GSI NP-treated mice.

FIG. 5D shows normal splenic architecture in GSI NP-treated mice (scale bar: 500 μm).

FIG. 6A diagrammatically illustrates an injection schedule for testing of the GSI NPs in non-alcoholic steatohepatitis (NASH) diet-fed mice.

FIG. 6B is a graph showing the GSI NPs' effect on glucose in the NASH diet-fed mice.

FIG. 6C is a graph showing the GSI NPs' effect on insulin in the NASH diet-fed mice.

FIG. 6D is a graph showing the GSI NPs' effect on body weight in the NASH diet-fed mice.

FIG. 6E is a graph showing the GSI NPs' effect on body composition in the NASH diet-fed mice.

FIG. 6F is a graph showing the GSI NPs' effect on liver and adipose tissue weights in the NASH diet-fed mice.

FIG. 6G is a graph showing the GSI NP-induced reduction in Notch target, fibrogenic and inflammatory gene expression.

FIG. 6H shows a reduction in liver fibrosis in the GSI NP-treated mice by quantification of Picrosirius red (scale bar: 500 μm); * p<0.05 as compared to control NP-treated mice, n=6-9/group.

FIG. 6I shows a reduction in liver fibrosis in the GSI NP-treated mice by F4/80+ staining (scale bar: 200 μm); ** p<0.01 as compared to control NP-treated mice, n=6-9/group.

FIG. 6J illustrates no increase in PAS+ intestinal goblet cells (scale bar: 50 μm) corresponding to FIGS. 6H and 6I.

FIG. 7A is a graph showing serum triglycerides in GSI NP-treated, high fat diet (HFD)-fed mice.

FIG. 7B is a graph showing liver triglycerides in GSI NP-treated, HFD-fed mice.

FIG. 7C is a graph showing serum cholesterol in GSI NP-treated, HFD-fed mice.

FIG. 7D is a graph showing liver cholesterol in GSI NP-treated, HFD-fed mice.

FIG. 7E and FIG. 7F are graphs showing plasma transaminases in GSI NP-treated, HFD-fed mice. n=11-12/group.

FIG. 8A is a photograph taken after four weeks of q3 day intraperitoneal injection of unencapsulated GSI (˜2.5 μ/kg/dose), showing hair graying in all GSI-treated animals but not in the controls.

FIG. 8B is a photograph showing hair graying in 80% of IV-injected GSI NP-treated animals. n=8-9, 11-12/group.

FIG. 9A is a graph showing serum triglycerides in GSI NP-treated, NASH diet-fed mice.

FIG. 9B is a graph showing liver triglycerides in GSI NP-treated, NASH diet-fed mice.

FIG. 9C is a graph showing serum cholesterol in GSI NP-treated, NASH diet-fed mice; *p<0.05.

FIG. 9D is a graph showing liver cholesterol in GSI NP-treated, NASH diet-fed mice; **p<0.01.

FIG. 9E and FIG. 9F are graphs showing plasma transaminases in GSI NP-treated, NASH diet-fed mice. n=6-9/group.

FIG. 10A diagrammatically illustrates an injection schedule for testing of the GSI NPs in NASH diet-fed mice.

FIG. 10B is a graph showing the effect of GSI NPs on glucose in the NASH diet-fed mice.

FIG. 10C is a graph showing the effect of GSI NPs on insulin in the NASH diet-fed mice.

FIG. 10D is a graph showing the effect of GSI NPs on body weight in the NASH diet-fed mice.

FIG. 10E is a graph showing the effect of GSI NPs on body composition in the NASH diet-fed mice.

FIG. 10F is a graph showing the effect of GSI NPs on liver and adipose tissue weights in the NASH diet-fed mice.

FIG. 10G is a graph showing reduction of the Notch target genes (Hes1, HeyL, Sox9, Spp1), markers of HSC activity (Colla1, Timp1) and inflammation (Cd45, Mcp1) in the NASH diet-fed mice.

FIG. 10H shows GSI NP-treated mouse livers with reduced fibrosis and inflammation as measured by Picrosirius red quantification; * p<0.05.

FIG. 10I shows GSI NP-treated mouse livers with reduced fibrosis and inflammation as measured by F4/80+ staining quantification; p<0.01.

FIG. 10J shows a lack of change in number of intestinal goblet cells corresponding to FIGS. 10H and 10I. Yellow arrows indicate examples of PAS+ cells; ***p<0.001 as compared to control NP-treated mice, n=6-9/group.

Similar reference characters denote corresponding features consistently throughout the attached drawings.

### BEST MODE(S)

The method of treating obesity-induced glucose intolerance and liver fibrosis includes administering to a patient a therapeutically effective amount of a substance for inhibiting the Notch signaling pathway of the patient. The substance is formed from encapsulated γ-secretase inhibitor (GSI) nanoparticles. As a non-limiting example, each nanoparticle may be formed from a GSI encapsulated in poly(lactic co-glycolic acid) (PLGA). The encapsulated GSI nanoparticles (GSI NPs) provide localized and effective inhibition of hepatic Notch signaling, thus improving obesity-induced glucose tolerance and liver fibrosis without intestinal side effects.

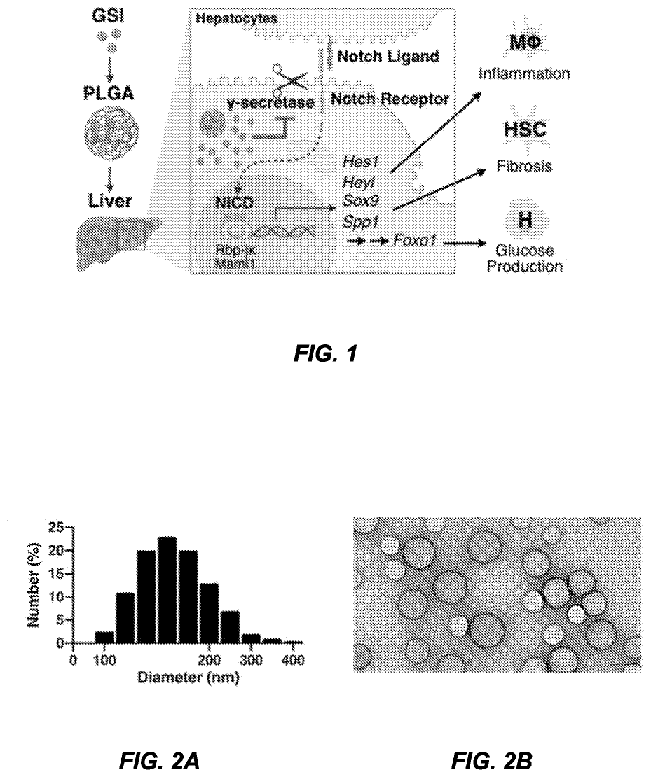

As illustrated in FIG. 1, GSI NPs accumulate in the liver, where they release GSI over time. Inhibition of γ-secretase prevents translocation of the Notch Intracellular Domain (NICD) into the nucleus, blocking target gene transcription. Downstream effects of Notch signaling inhibition include reduced macrophage (MΦ)-mediated inflammation, hepatic stellate cell (HSC)-induced fibrosis and hepatocyte (H) glucose production.

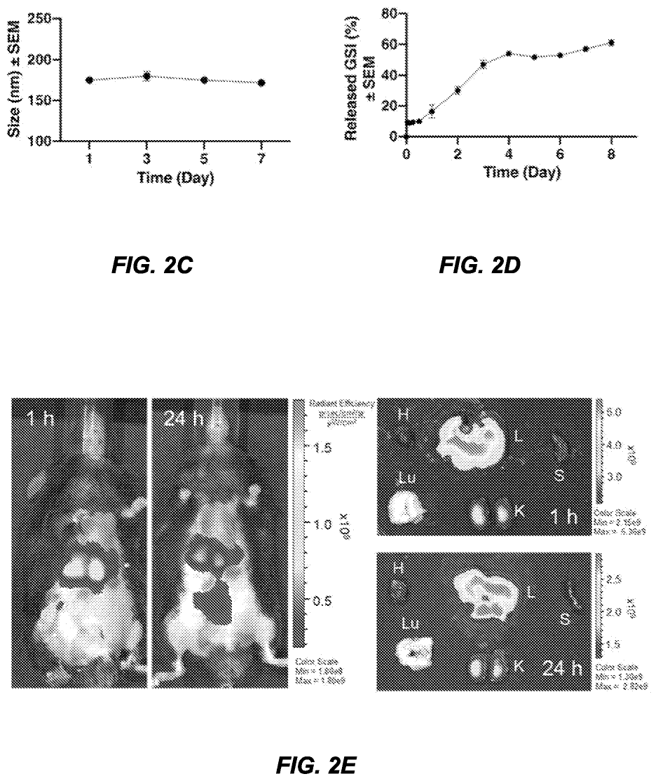

The GSI NPs were prepared using an emulsion/solvent evaporation method to encapsulate dibenzazepine (a bioavailable γ-secretase inhibitor) in a PLGA matrix. Dibenzazepine and PLGA were dissolved in a dichloromethane (DCM) and 3% poly(vinyl alcohol) (PVA) solution. Empty (control) NPs were prepared using the same method without a drug. The PLGA mixture was sonicated and dispersed into a 0.3% PVA solution and the DCM was evaporated. The synthesized NPs, labeled with a Cy5.5 fluorophore, were collected by centrifuge. Resultant spherical NPs were ˜180 nm in diameter as determined by dynamic light scattering, and size remained relatively constant over seven days, as shown in FIGS. 2A, 2B, 2C and 2D.

The GSI NPs had a loading capacity of 8.9±0.01%, and an encapsulation efficiency of 88.6±0.1%. To monitor the dissociation behavior and release kinetics of GSI, the NPs were loaded with 24 μg of GSI and incubated in phosphate buffered solution (PBS) (pH 7.4) containing 20% ethanol at 37° C. The NPs gradually disassociated due to the biodegradation of PLGA, triggering the encapsulated cargo release. This dissociation and sustained release occurred over eight days as measured by the concentration of GSI in the buffer. In vivo, at 1 h and 24 h after intravenous (IV) tail vein injection, nanoparticles were distributed primarily in the liver and kidneys, reflective of accumulation in the liver and renal excretion, as shown in FIG. 2E.

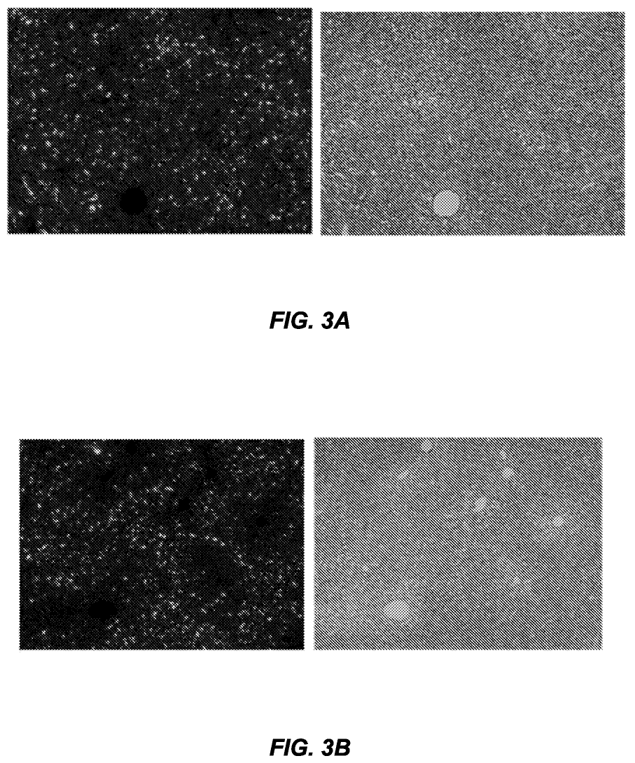

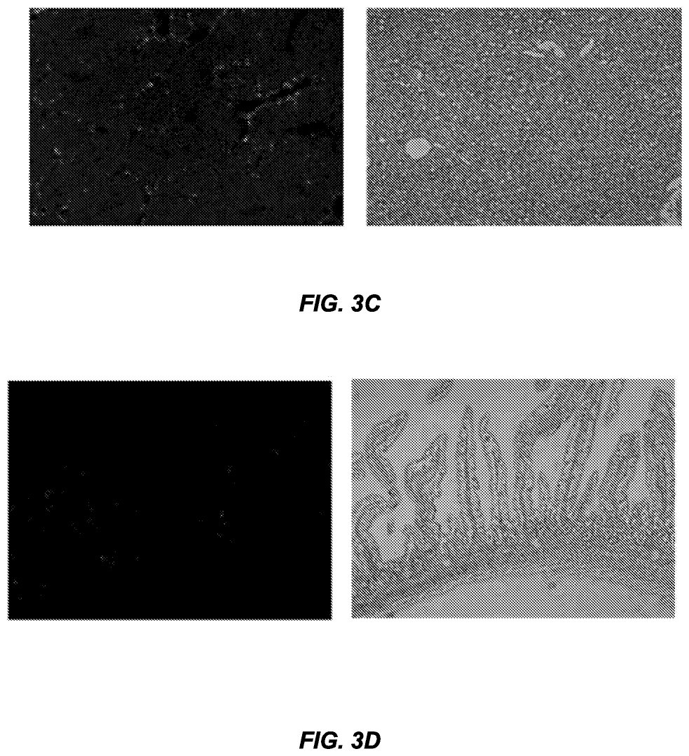

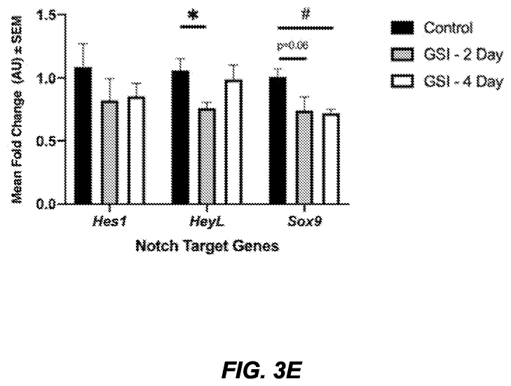

To assess Notch inhibition efficacy in vivo, a single dose (5 mol/kg, —2.5 mg/kg) of GSI NPs suspended in normal saline was administered to C57BJ/6 male mice IV via tail vein. GSI NPs were directly visualized in the liver, as shown in FIGS. 3A, 3B and 3C, but not the GI tract, as seen in FIG. 3D. Hepatic expression of Notch target genes was then analyzed, chosen empirically as targets that track with hepatocyte Notch activity in vitro and in vivo, at multiple time points post-treatment. Reduced liver Notch target gene expression at two and four days was found, as shown in FIG. 3E.

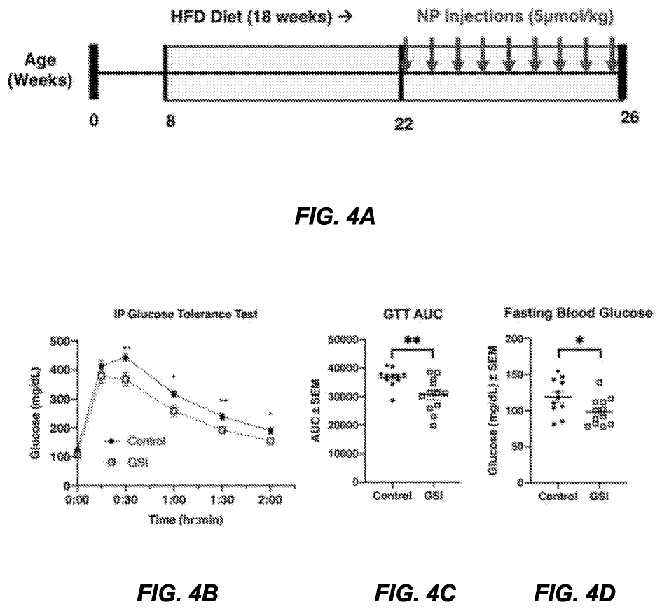

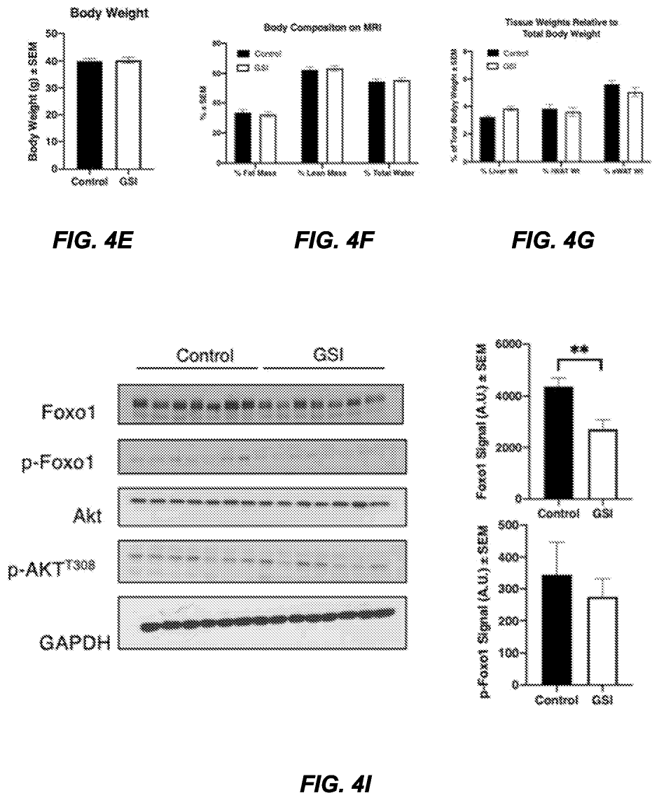

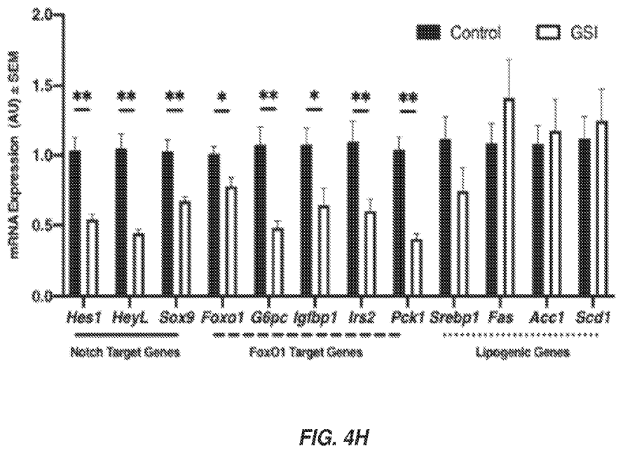

The GSI NPs were applied to a mouse model of diet-induced obesity (DIO) and glucose intolerance/insulin resistance. Eight-week-old C57BL/6 male mice were fed HFD (60% kcal from fat) for a total of 18 weeks, as shown in FIG. 4A. After 14 weeks of diet-feeding, animals were randomized into two groups of equal body weight. GSI or vehicle (control) NPs suspended in PBS was administered by tail vein twice weekly for four weeks. GSI NP-treated mice showed significant improvements in glucose tolerance after a 2 g/kg glucose load with a 16.5% reduction in area under curve (AUC) compared to the control NP-treated mice, as shown in FIGS. 4B and 4C. At sacrifice, normalized fasting glucose levels after a 16 h fast were observed (a parameter in diagnosing diabetes) in the GSI NP-treated group, from ˜120 mg/dL in control to ˜98 mg/dL in GSI-treated animals (see FIG.

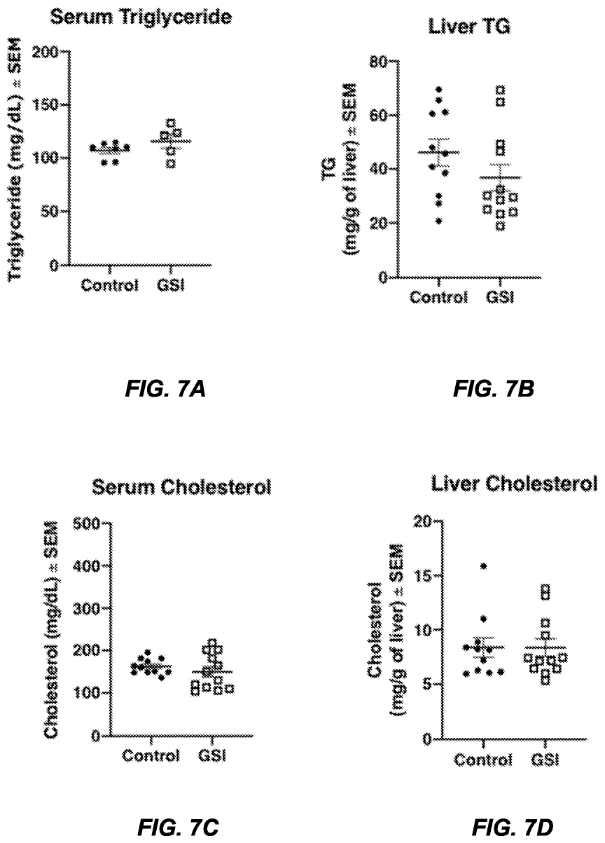

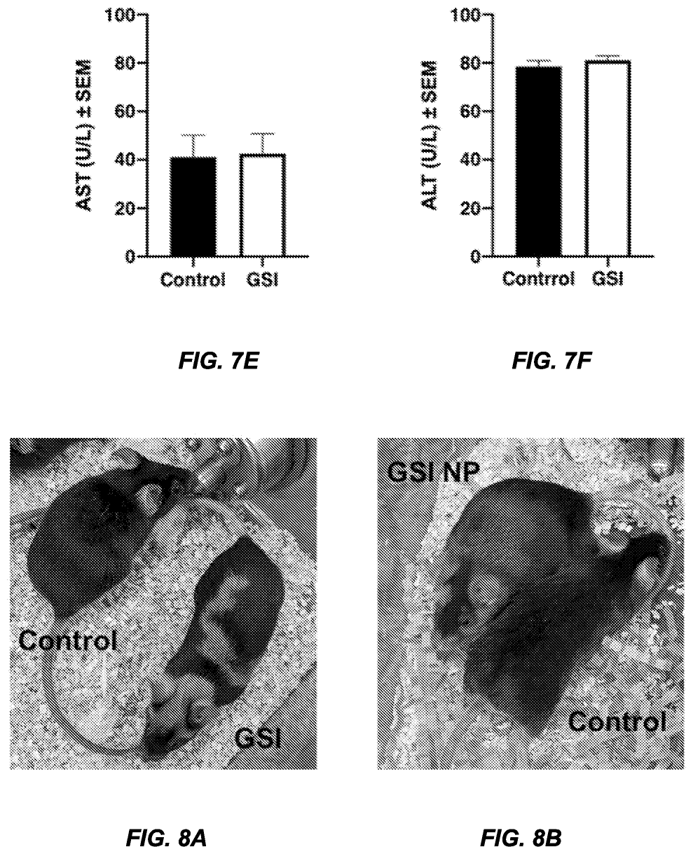

Consistently, reduced fasting glucose levels in GSI NP-treated mice was observed (FIG. 4D), despite similar body fat as measured by magnetic resonance imaging (MRI), commensurate with unchanged body, liver, inguinal white adipose tissue (iWAT), and epididymal white adipose tissue (eWAT) weights (see FIGS. 4E, 4F and 4G). No significant differences in liver lipid content or transaminases (aspartate transaminase [AST] and alanine transaminase [ALT]) were observed, and only trivial differences were observed in serum lipids, as shown in FIGS. 7A-7F. These data suggest that GSI NP treatment improves glucose tolerance, but not due to alterations in body weight, adiposity, or liver lipid content/injury.

To assess the causal pathways for improved glucose tolerance, the fresh frozen whole liver was analyzed by qPCR. As expected, GSI NPs reduced Notch target gene expression by ˜34-58%. Consequently, a significant reduction in FOXO1 expression and multiple FOXO1 target genes were observed, including those associated with gluconeogenesis (G6pc, Pck1) (see FIG. 4H). There was no corresponding reduction in lipogenesis gene expression. Western blot similarly demonstrated a significant ˜38% decrease in FOXO1 in GSI NP-treated mice compared to controls, with a proportional reduction in phospho-FOXO1 (p-FOXO1) levels. There was also no significant difference in phospho-Akt (p-Akt) levels between groups (see FIG. 4I). Taken together, these data suggest that GSI NP-induced amelioration of obesity-induced glucose tolerance is due to reduced FOXO1-mediated HGP.

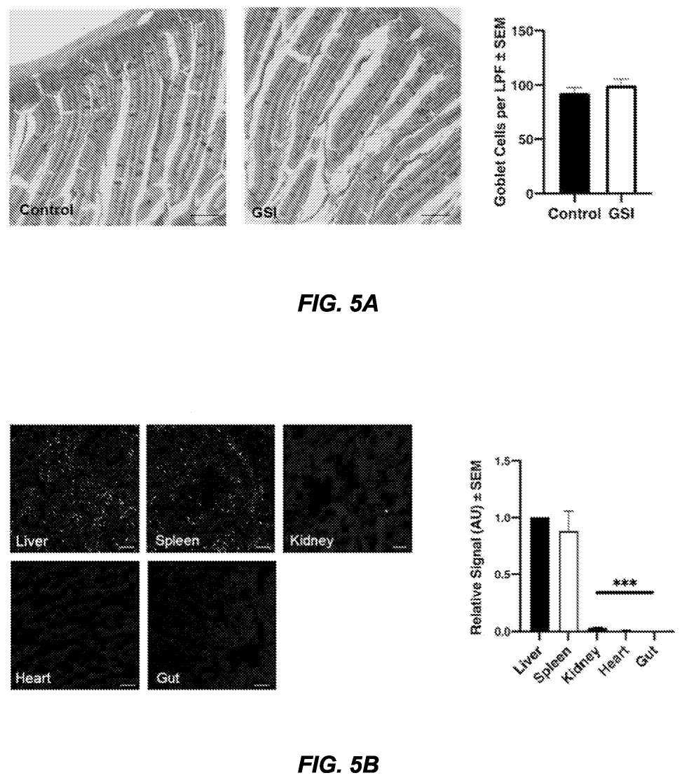

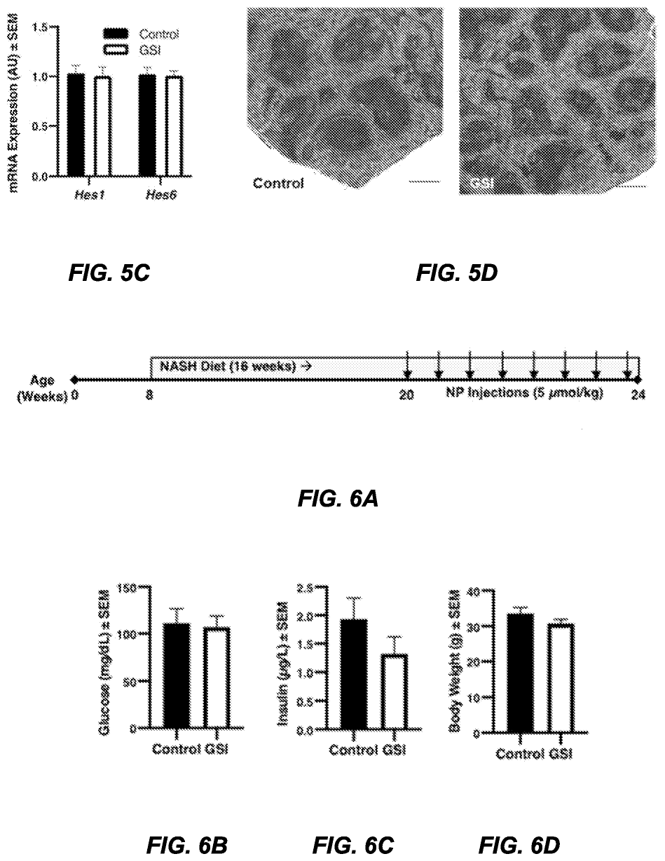

Mild hair graying in 80% of GSI NP-treated mice was also observed, although subjectively less than in mice injected with unencapsulated GSI (see FIGS. 8A and 8B). Hair graying has also been noted in clinical trials of oral GSI for Alzheimer's Disease, suggestive of dermal distribution of GSI regardless of method of administration. Two other known GSI side effects were also investigated: intestinal metaplasia and marginal zone atrophy in the spleen (i.e., de-differentiation of B-cells in the spleen). The minimal Cy5.5 signal in the small intestine of both control and GSI NP-treated mice was visualized. Consistently, the goblet cell number was unchanged in the GSI NP-treated group (see FIG. 5A). This is in sharp contrast to mice treated with unencapsulated GSI at a similar dose, which show a threefold increase in goblet cells, leading to mucinous diarrhea, dehydration, weight loss, and eventually death.

As expected based on NP size, the Cy5.5 signal was observed in the spleen, but no other extrahepatic tissues, indicating the presence of the NP shell (see FIG. 5B). Though drug concentration was not directly measured, unchanged splenic Notch activity was observed, as assessed by expression of the most highly abundant Hes/Hey genes in spleen (Hes1, Hes6), in GSI NP-treated animals (see FIG. 5C). Consistently, there was no detection of differences in splenic architecture in representative samples analyzed in a blinded fashion (see FIG. 5D). Thus, the Cy5.5 signal in the spleen may represent NP shells scavenged by circulating monocytes, or accumulative entrapment of a small percentage of NPs over time, as the spleen did not demonstrate significant signal in the initial 24 h after injection (see FIG. 5e). Together, these data suggest that while GSI NPs may circulate to other organs through systemic circulation, the majority of drug action occurs in the liver, as intended. Other tissues examined, including kidney and heart, did not demonstrate significant Cy5.5 signal (see FIG. 5A).

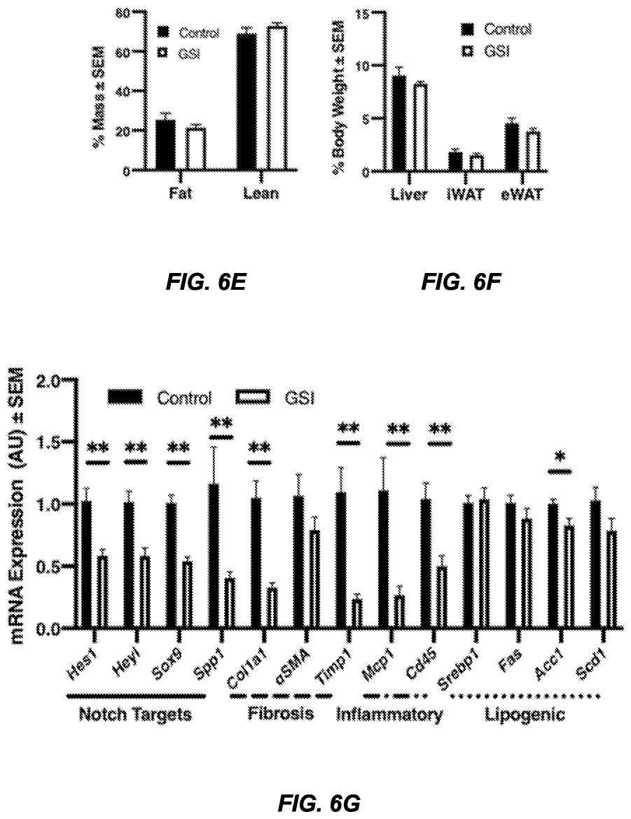

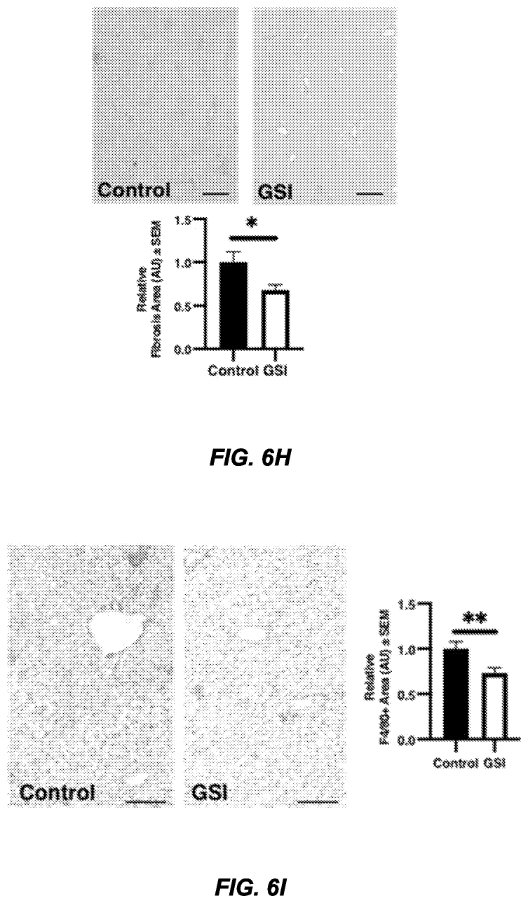

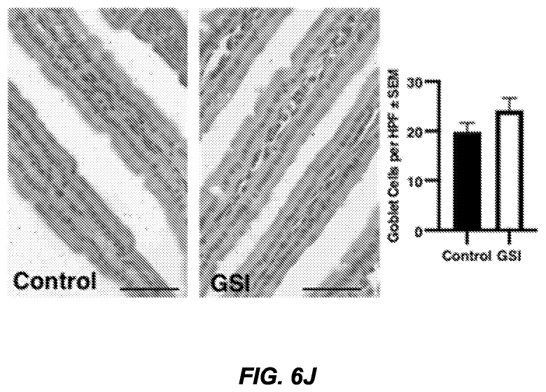

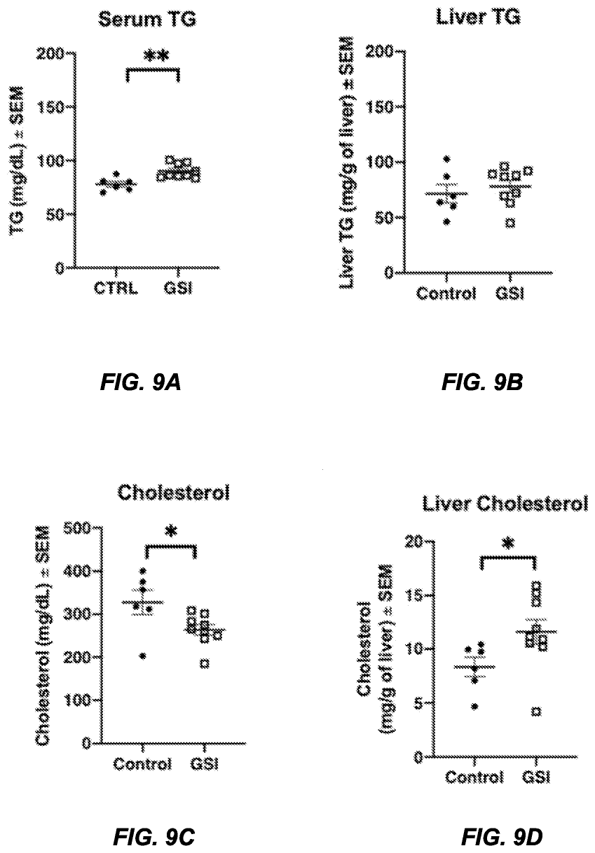

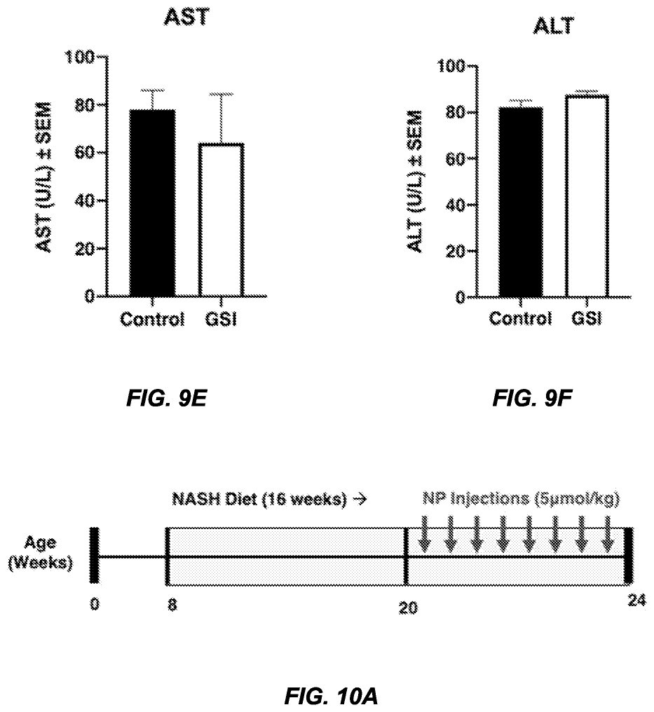

Aberrant hepatocyte Notch activity in patients with NASH, or mice fed a NASH-provoking diet enriched in fructose, palmitate, and cholesterol with ad libitum access to fructose-containing drinking water, has been observed. Systemic GSI treatment reduces liver fibrosis in these mice, but with significant GI toxicity. To test safety and efficacy of GSI NPs to ameliorate NASH-induced liver fibrosis, GSI or vehicle (control) NPs was applied twice weekly for four weeks to weight-matched, NASH diet-fed C57BL/6 male mice (see FIG. 6A). No differences in body weight, body composition, or tissue weights were observed between groups (see FIGS. 6B, 6C, 6D, 6E and 6F). Further, and consistent with phenotypes from hepatocyte-specific Notch loss-of-function mice and GSI NP-treated HFD-fed mice, trivial changes in serum or liver lipids and serum transaminases were observed (see FIGS. 9A-9F). Nevertheless, GSI NPs reduced Notch target gene expression with a parallel and significant reduction in inflammatory and fibrogenic gene expression (see FIG. 6G). Consistently, a 32% reduction in liver collagen staining (FIG. 6H), and a 27% reduction in liver macrophage number (FIG. 6I), were observed, contributing to both liver inflammation and fibrosis. All GSI NP-treated animals had mild hair graying, but showed no increase in intestinal goblet cell number as compared to control (see FIG. 6J). Overall, these data suggest that GSI NPs can ameliorate NASH diet-induced liver fibrosis and inflammation without the intestinal toxicity of unencapsulated GSI treatment.

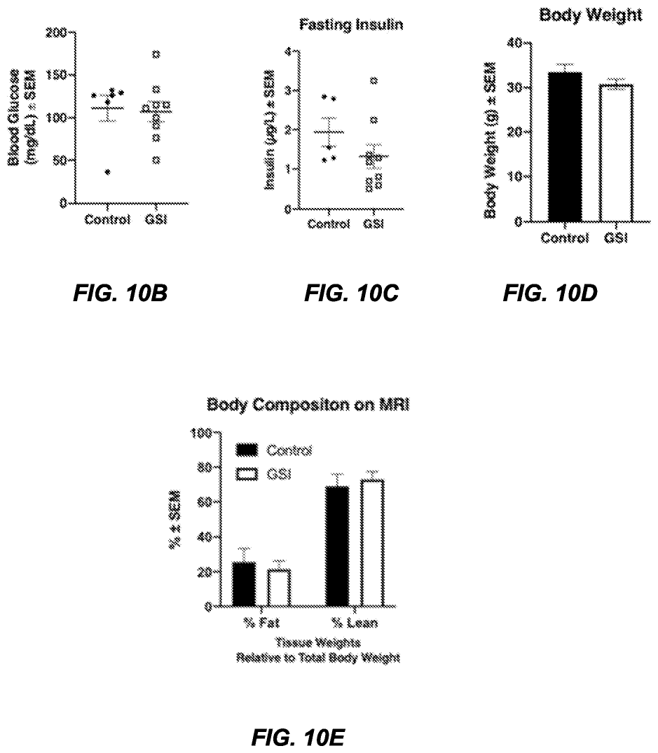

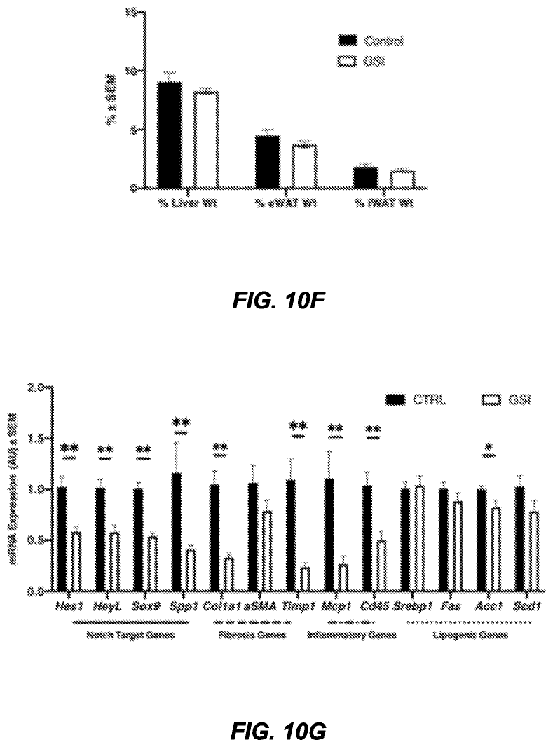

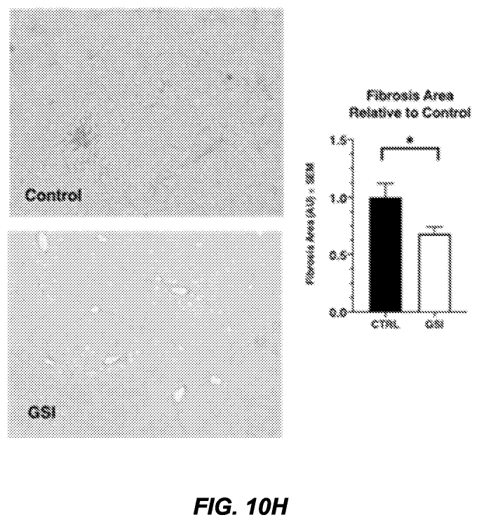

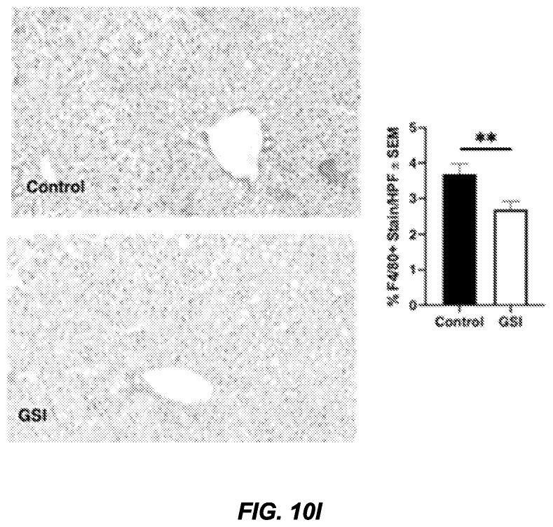

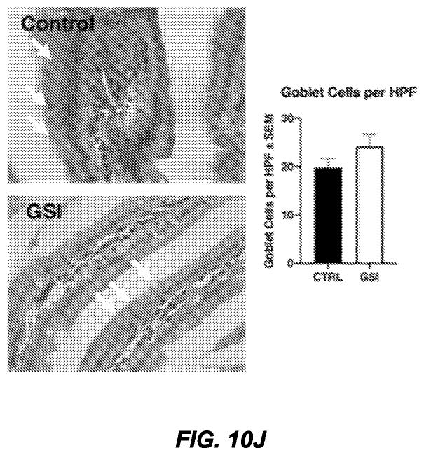

Hepatocyte Notch reactivation is a distinguishing feature in NASH. Similar to data with glucose tolerance, reduced hepatic fibrosis was observed with naked GSI treatment in a mouse NASH model, but with significant GI toxicity. Thus, GSI NPs were administered to C57BL/6 male mice fed a NASH-inducing diet enriched for fructose, palmitate, and cholesterol with ad libitum access to fructose-containing drinking water (see FIG. 10A). After 12 weeks of this diet, the NASH diet-fed animals were body weight matched and randomized to receive GSI or vehicle (control) NPs, twice weekly for four weeks. There were no differences in body weight, body composition, or tissue weights between groups (see FIGS. 10B-10E). Further, and consistent with phenotypes from hepatocyte-specific Notch loss-of-function mice and GSI NP-treated HFD-fed mice, trivial changes in liver lipids and serum transaminases were observed, and a small but significant reduction in serum cholesterol (see FIGS. 9A-9F). Nevertheless, GSI NPs reduced Notch target gene expression (see FIG. 10G), with a parallel and significant reduction in inflammatory and fibrogenic gene expression, suggestive of decreased hepatic stellate cell (HSC) activity.

Consistently, a significant 32% reduction in fibrosis in the GSI NP group relative to control was observed by polarized light microscopy of liver Picrosirius Red staining (see FIG. 10H). Commensurate with these findings, there was a 37% reduction in F4/80+ staining in the liver, a marker of Kupffer cell infiltration (see FIG. 10I). Kupffer cells are thought to be critical inflammatory mediators of NAFLD and NASH development through cytokine release and activation of HSCs. Finally, though the GSI NP-treated animals had hair graying, as with the DIO insulin resistance model, no Cy5.5 signal was visualized in the small intestine, nor a significant difference between control-treated and GSI NP-treated mice in intestinal metaplasia (see FIG. 10J). Overall, these data suggest that GSI NPs can reverse NASH-induced fibrosis, without the obvious intestinal toxicity that plagues unencapsulated GSIs.

The method of treating obesity-induced glucose intolerance and liver fibrosis provides liver PLGA NP uptake to block pathologic hepatocyte Notch signaling, while reducing the toxic profile of traditional GSI administration. GSI NPs reduce HFD-induced glucose intolerance as well as hepatic fibrosis and inflammation in a dietary NASH mouse model. These encouraging effects are consistent with systemic Notch signaling inhibition, but circumvent intestinal and splenic toxicity. Though serum concentrations of GSI were not compared directly, the known comparable efficacy in metabolic endpoints suggests that NP administration delivers therapeutic doses of GSI to intended targets while reducing or avoiding toxicity in other tissues. These data provide proof-of-concept that nanomedicine allows for repurposing of a previously intolerable medication.

The PLGA nanoparticles were prepared with an emulsion/solvent evaporation method. Briefly, 1 mg of dibenzazepine and 10 mg of PLGA were dissolved in 0.8 mL of DCM, followed by the addition of 2 mL of 0.3% PVA solution. This mixture was then sonicated using a probe sonicator (amplitude 30, 15 sec on, 15 sec off×10 min) and dispersed into 8 mL of 0.3% PVA solution under stirring. Finally, DCM was evaporated using a rotary evaporator. GSI NPs were labeled by adding 0.5% Cy5.5 fluorophore and PLGA (w/w) while dissolving the dibenzazepine and PLGA. Empty (control) NPs were fabricated using the same method without drug. The synthesized NPs were collected by centrifuge at 13,000 rpm for 45 minutes. The resultant NPs were spherical in shape and ˜180 nm in diameter. NPs were then analyzed for loading capacity (LC) and encapsulation efficiency (EE). LC and EE of GSI were determined by high-performance liquid chromatography (HPLC) using an Agilent C18 column (4.6×50 mm) eluted with water and acetonitrile (starting at 95:5 and then after 6 min, gradient up to 5:95; wavelength 254 nm). LC and EE were calculated as LC=B/C×100% and EE=B/A×100%, where A equals the expected encapsulated amount of GSI using a standard curve, B equals the encapsulated amount of GSI, and C equals the total weight of the particles. Particle size and polydispersity intensity were measured by dynamic light scattering. The zeta potential of the NPs was determined by their electrophoretic mobility after dilution in DI water using the same instrument. NP morphology was assessed by a field-emission scanning electron microscope.

The in vitro release profile of the GSI-loaded PLGA nanoparticles was evaluated through incubation of NPs in 25 mL PBS buffer (NaCl, 137 mM; KCl, 2.7 mM; Na₂HPO₄, 10 mM; KH₂PO₄, 2 mM; pH 7.4) containing 20% ethanol at 37° C. on an orbital shaker (100 rpm/min). At predetermined time points, the buffer sample was taken for HPLC analysis (C18 reverse phase column 4 6 m×15 cm, wavelength: 254 nm) of GSI with the mobile phase composed of water and acetonitrile (starting at 95:5, then after 6 min, gradient up to 5:95, then back to 95:5 in 2 min, n=3).

Unless otherwise specified, seven-week old male C57BL/6 mice were obtained from Jackson Labs (Bar Harbor, Me.). All animals were treated in accordance with the Guide for Care and Use of Laboratory Animals and protocols were approved by the Columbia Institutional Animal Care and Use Committee (IACUC). Mice were caged at 22±1° C. with free access to water and diet on a 12 h light/dark cycle unless otherwise specified. Mice were adapted to their environment for at least one week prior to starting experiments.

For in vivo studies of the GSI NPs in lean mice, male C57BL/6 mice were randomized into four groups (n=5/group) and treated with empty PLGA NPs or GSI NPs (5 μmol/kg) injected once via tail vein. Animals were injected either two, four, or seven days prior to sacrifice (control group was injected 2 days prior) by CO₂.

Male C57BL/6 were fed either a normal chow diet, a high-fat diet (60% kcal from fat) or a NASH provoking-diet with ad libidum access to sucrose-fructose containing drinking water. Four weeks prior to sacrifice, animals were randomized to control or GSI NPs (5 μmol/kg, —2.5 mg/kg) injected twice weekly (q 3-4 days) by tail vein injection. The HFD-fed group received a total of nine doses over the course of the experiment (45 μmol/kg, —22.5 mg/kg) and the NASH diet-fed group received eight doses total (40 μmol/kg, —20 mg/kg). MRI was performed immediately prior to the final injection. After four weeks of treatment, the animals were fasted for 16 h (overnight) and blood glucose and insulin levels were obtained. The animals were then euthanized by CO₂ inhalation, and blood, liver, kidney, splenic, cardiac, and adipose tissues (iWAT and eWAT) were collected for analyses. Blood samples were allowed to coagulate prior to collecting serum. At sacrifice, animals were euthanized by CO₂ inhalation followed by cervical dislocation, and blood, liver, kidney, spleen, heart and adipose tissues (iWAT and eWAT) collected for analyses. Blood samples coagulated for 15 min prior to collecting serum.

After eight NP treatments, mice were fasted for 16 h (overnight) before intraperitoneal injection of 2 g/kg glucose solution. Blood glucose levels were monitored every 30 min for 120 min by tail vein. Snap-frozen liver, kidney, heart, and intestine sections were fixed with 4% paraformaldehyde at 4° C. for 48 hours prior to dehydration with 30% sucrose solution for 72 hours at room temperature. Frozen tissue specimens were embedded in optimal cutting temperature (OCT) cutting media and mounted in 5 μm thickness sections. Direct visualization of Cy5.5 signal from the nanoparticles was performed using fluorescent microscopy and quantified over background (as measured by tissue fluorescence from non-injected animals, n=4).

For the in vivo studies of the GSI NPs in diet-induced obese mice with hepatic steatosis and fibrosis, male C57BL/6 mice were fed a high cholesterol, palmitate, and fructose containing diet and given ad libidum access to sucrose-fructose containing drinking water for 16 weeks to induce obesity and hepatic fibrosis. At 12 weeks of diet, the animals were randomized into two groups (n=6, 9) and treated with empty PLGA NPs or GSI NPs (5 μmol/kg), injected twice weekly IV (q 3-4 days) via tail vein. Body weight was monitored weekly during the treatment period. MRI was performed immediately prior to the final injection.

After four-weeks' treatment, the animals were fasted for 4 h and blood glucose and insulin levels were obtained. The animals were then euthanized by CO₂ inhalation, and blood, liver, kidney, and adipose tissues (iWAT and eWAT) were collected for analyses. Blood samples were allowed to coagulate prior to collecting serum.

For the visualization of the NPs, snap frozen liver, kidney, heart, and intestine sections were fixed with 4% paraformaldehyde at 4° C. for 48 hours prior to dehydration with 30% sucrose solution for 72 hours at room temperature. Specimens were then embedded in OCT compound and mounted in 5 um thickness sections. Direct visualization of Cy5.5 signal from the nanoparticles was done using fluorescent microscopy. Representative images were taken using a Zeiss light microscope coupled with an AxioCam MR3 camera.

Serum triglycerides and cholesterol were measured according to manufacturer's instructions using colorimetric assays. Serum AST and ALT were measured per manufacturer's instructions using standard colorimetric or kinetic assays. Serum insulin levels were measured per manufacturer's instructions. Liver lipids were extracted using the Folch method. In brief, —100 mg of snap frozen liver tissue was homogenized in lx PBS. Sample was added to a chloroform-methanol mixture and the aqueous layer evaporated under N2 gas. Liver triglycerides were diluted 1:10 prior to colorimetric assay.

RNA was extracted from liver and spleen with Trizol (Invitrogen) prior to cDNA synthesis and quantitative PCR with a DNA Engine Opticon 2 System (Bio-Rad) and GoTaq qPCR Master Mix (Promega A600A). mRNA levels were normalized to Rp123 mRNA using the AAC(t) method and are presented as relative transcript levels to control. Primer sequences are available upon request.

Paraffin fixed sections of the spleen were fixed with 10% formaldehyde for 48 h and stored in 70% ethanol for 72 h at room temperature prior to mounting in 5 μm thickness sections and hematoxylin and eosin staining. Images of the entire specimen were sent to a blinded pathologist (ELM) and examined based on the presence or absence of marginal zone atrophy. Paraffin fixed sections of the liver were fixed with 10% formaldehyde for 48 h and stored in 70% ethanol for 72 h at room temperature prior to mounting in 5 μm thickness sections. For each liver, 12-18 images of non-overlapping Picrosirius Red-stained regions were used to quantify collagen deposition under polarized light microscopy using ImageJ (NIH) software. Snap frozen liver sections were fixed with 4% paraformaldehyde at 4° C. for 48 h prior to dehydration with 30% sucrose solution for 72 h at room temperature. Specimens were then embedded in OCT compound and mounted in 5 μm thickness sections. For each liver, 12-18 images of non-overlapping F4/80-stained regions were examined under brightfield microscopy and analyzed by ImageJ (NIH) software using thresholding and area measurement. Paraffin fixed sections of small intestine were fixed with 10% formaldehyde for 48 h and stored in 70% ethanol solution for 72 h at room temperature. Periodic acid-Schiff (PAS) stained sections of intestine were examined under brightfield microscopy and goblet cell numbers were quantified using ImageJ (NIH) software as previously described.

For the quantification of F4/80 staining, fresh frozen liver sections were fixed with 4% paraformaldehyde at 4° C. for 48 h prior to dehydration with 30% sucrose solution for 72 h at room temperature. Specimens were then embedded in OCT compound and mounted in 5 μm thickness sections. For each liver, 12-18 images of non-overlapping F4/80-stained (Cell Signaling Technology [CST] #70076, 1:500 dilution) regions were examined under brightfield microscopy and analyzed in ImageJ (NIH) software using thresholding and area measurement.

For the quantification of goblet cell number, paraffin fixed sections of the liver were fixed with 10% formaldehyde for 48 h and stored in 70% ethanol solution for 72 h at room temperature. Periodic acid-Schiff (PAS) stained sections of intestine were examined under brightfield microscopy and goblet cell numbers were quantified using ImageJ (NIH) software.

With regard to detection of the Cy5.5 signal, snap frozen liver, kidney, heart, and intestine sections were fixed with 4% paraformaldehyde at 4° C. for 48 hours prior to dehydration with 30% sucrose solution for 72 hours at room temperature. Specimens were then embedded in OCT compound and mounted in 5 μm thickness sections. Representative images were taken using a Zeiss light microscope coupled with an AxioCam MR3 camera (Carl Zeiss).

For the Western blots and quantification of the signal, fresh frozen liver tissue was lysed in a Triton-based lysis buffer to obtain whole-cell lysate. Protein was loaded into a 10% SDS-PAGE gel Immunoblots were conducted on seven randomly chosen samples per group and incubated with primary antibodies (1:1000 dilution unless otherwise specified) against Akt (CST #9272), p-Akt^{T308} (CST #4056), FOXO1 (CST #2880), p-FOXO1^{S256} (CST #84192), and GAPDH conjugated with horseradish peroxidase (Proteintech HRP-60004, 1:5000 dilution). Quantification of signal was performed using ImageJ software.

Differences between groups were analyzed using the two-tailed unpaired Student's t-test assuming unequal variance with p-value <0.05 to determine significance. All results are presented as mean±standard error of the mean (SEM). Statistical analysis was performed using Prism software (GraphPad).

It is to be understood that the method of treating obesity-induced glucose intolerance and liver fibrosis is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A method of treating obesity-induced glucose intolerance and liver fibrosis, comprising the step of administering to a patient a therapeutically effective amount of a substance for inhibiting a Notch signaling pathway of the patient, the substance comprising encapsulated γ-secretase inhibitor nanoparticles.

2. The method of treating obesity-induced glucose intolerance and liver fibrosis as recited in claim 1, wherein said encapsulated γ-secretase inhibitor nanoparticles are prepared by encapsulating γ-secretase inhibitor in poly(lactic co-glycolic acid).

3. The method of treating obesity-induced glucose intolerance and liver fibrosis as recited in claim 2, wherein said encapsulated γ-secretase inhibitor nanoparticles are prepared by encapsulating γ-secretase inhibitor nanoparticles in poly(lactic co-glycolic acid).

4. The method of treating obesity-induced glucose intolerance and liver fibrosis as recited in claim 3, wherein the γ-secretase inhibitor nanoparticles comprise dibenzazepine.

5. A composition for treating obesity-induced glucose intolerance and liver fibrosis, comprising encapsulated γ-secretase inhibitor nanoparticles.

6. The composition for treating obesity-induced glucose intolerance and liver fibrosis as recited in claim 5, wherein said encapsulated γ-secretase inhibitor nanoparticles comprise γ-secretase inhibitor nanoparticles encapsulated in poly(lactic co-glycolic acid).

7. The composition for treating obesity-induced glucose intolerance and liver fibrosis as recited in claim 5, wherein the γ-secretase inhibitor nanoparticles comprise dibenzazepine.

8. The composition for treating obesity-induced glucose intolerance and liver fibrosis as recited in claim 6, wherein the γ-secretase inhibitor nanoparticles comprise dibenzazepine.

9. A method of inhibiting the Notch signaling pathway of a patient, comprising the step of administering to the patient a therapeutically effective amount of the composition of claim 6.

10. A method of inhibiting the Notch signaling pathway of a patient, comprising the step of administering to the patient a therapeutically effective amount of the composition of claim 8.
